(19) United States
(12) Patent Application Publication
(10) Pub. No.: US 2023/0414344 A1
(43) Pub. Date: Dec. 28, 2023

(54) TWO-PIECE INTRAOCULAR LENSES WITH SHAPE-CHANGING OPTIC

(71) Applicant: JelliSee Ophthalmics Inc., McLean, VA (US)

(72) Inventor: Forrest J. Ellis, McLean, VA (US)

(21) Appl. No.: 18/337,542

(22) Filed: Jun. 20, 2023

Related U.S. Application Data
(60) Provisional application No. 63/354,473, filed on Jun. 22, 2022.

(51) Int. Cl. A61F 2/16

(52) U.S. Cl. CPC A61F 2/1651; A61F 2/1635; A61F 2002/1682; A61F 2002/169; A61F 2002/16901; A61F 2230/0058

(57) ABSTRACT

A two-piece intraocular lens (IOL) with an anterior shape-changing optic is provided. The shape-changing interchangeable optic includes an elastic anterior face located anterior to the equator and comprising anterior arms releasably connected to actuating haptics of a base.

### CROSS-REFERENCE TO RELATED APPLICATION

The present application claims priority to U.S. Provisional Patent Application No. 63/354,473 filed on Jun. 22, 2023, which is incorporated by reference in its entirety.

### TECHNICAL FIELD

The present disclosure relates to an accommodative two-piece intraocular lens that includes an optic that can change shape as the intraocular lens transitions from an accommodative state to a dis-accommodative state.

### BACKGROUND

The lens of the eye constitutes cells arranged in a lamellar manner and is divided into a central nucleus and a peripheral cortex. The lens is enclosed by a cellular basement membrane (the lens capsule). Along with the cornea, the lens focuses light (refraction) onto the retina of the eye. Refractive power is measured in diopters. The lens contributes approximately a third of the refractive power of the eye and it is responsible for fine-tuning the eye's focus so that objects over a wide range of distances can be seen clearly. The process whereby the lens changes the focus of the eye is called accommodation. Accommodation is measured in diopters of accommodation change. Accommodation occurs with contraction of the ciliary muscle, which reduces force on the lens suspensory zonular fibers (zonules). The lens zonules extend from the ciliary muscle to the near equatorial regions of the lens and suspend the lens behind the iris. Reduction of force on the zonules allows the lens of the eye to assume its natural, less stressed, more spherical shape; thereby increasing its dioptric power, and this brings objects at near into focus. The change in focus from distance focus to near focus is generally smooth and controlled by a complex neural feedback mechanism. The relative contribution to accommodation of the lens zonules, the lens capsule, the lens nucleus and the surrounding lens cortex is the subject of ongoing research.

With age, there is a gradual reduction in the ability of the lens to alter the focus of the eye (presbyopia). This manifests in a gradual loss of capacity to focus on near objects, typically requiring optical assistance (e.g. reading glasses, bifocals) beginning around the fifth to sixth decades of life. There are many theories explaining accommodation and the loss of accommodation. It is generally accepted that with aging the lens substance itself becomes more resistant to deformational change (e.g. less pliable, more rigid, or firmer). A comprehensive and definitive understanding of accommodation and the cause(s) of presbyopia is still subject to scientific inquiry. However, much is already known regarding structural and functional changes of the lens and the other structures of the eye that contribute to accommodation and to the gradual loss of accommodation.

The ciliary muscle has been shown to function throughout life without significant loss of function with aging. The lens zonules include anterior fibers which attach to the lens anterior to the lens equator, equatorial fibers which attach at the lens equator, and posterior fibers which attach posterior to the lens equator. It has been demonstrated that the anterior fibers primarily alter the shape of the anterior lens surface and the posterior fibers primarily control the shape of the posterior lens surface. The sparse and less robust equatorial fibers play a lesser role in changing of the shape of the surfaces of the lens. Some conditions and genetic disorders may contribute to lens zonule loss or breakage, but in general the lens zonules remain functional throughout life. Therefore, changes in the lens zonules are not considered a significant factor in the loss of accommodation.

The lens nucleus and cortex are unique in that the cellular makeup and structure of the lens allows for reshaping of individual cells with intracellular flow of cytosol (the fluid within the cell). As a result, the individual cell shape can change and cumulatively the entire lens contents, contained within the lens capsule, can be considered a viscoelastic or “semi-flowable fluid”. However, in youth, the lens nucleus is less resistant to deformational change (e.g. less firm or more pliable), than compared to the lens cortex. This is termed the lens elastic gradient. Studies have shown that with aging the lens nucleus becomes more resistant to deformational change (e.g. less pliable, or firmer). Likewise, studies show that the lens cortex also becomes more resistant to deformational change (e.g. less pliable or firmer with aging). However, the lens nucleus becomes firmer at a faster rate than the lens cortex. The crossover, where the nucleus becomes firmer, less pliable, or more resistant to deformational change than the lens cortex occurs around age 40-50 years of age. This relative change in the difference in firmness of the nucleus relative to that of the cortex correlates with the loss of accommodation and the onset of presbyopia.

The human crystalline lens can be affected by one or more disorders or conditions that reduces its function and/or reduces the clarity of the lens. A common condition that occurs with aging is the gradual opacification and reduced transparency of the lens of the eye. This condition is termed a cataract. Surgical removal of a cataractous lens and placement of an artificial replacement lens (such as an intraocular lens (“IOL”)) within the eye is a common surgical procedure. The development of a suitable IOL that can provide the optical quality and accommodation provided by the youthful biological lens has not been developed.

There are generally two classes of IOLs that have been developed that attempt to overcome the lack of accommodation of an IOL used to replace the natural lens when cataract surgery is performed: pseudo-accommodating lenses and accommodating lenses. A pseudo-accommodating lens can be a multiple focal point lens that uses a ring for distance focus and one or more center optics for intermediate and near focus. Other designs use diffraction optics to obtain a range of focus or use optics to achieve an extended depth of focus (EDOF). Multi-focus optics, diffraction optics, and EDOF optic IOLs can result in disruptive optical aberrations such as glare, halos, reduced contrast sensitivity, etc. Centration of these lenses within the capsular bag is important to their best visual function. These lenses use non-deforming optical elements and do not achieve the visual quality of a natural, youthful lens of the human eye. The accommodating class of IOLs includes a silicone elastomeric hinged lens that allows forward movement of the optic when the eye focuses at near. These lenses are typically placed in the lens capsular bag (the remaining thin layer of basement membrane that is the outermost layer of the natural lens and is typically left in place when the contents of the lens are removed during cataract surgery). Due to progressive fibrosis and stiffening of the lens capsule following cataract removal, the effective accommodation with these lenses is known to diminish over time.

Overall, these lenses may be adequate for distance and intermediate vision, but only provide accommodation of about two diopters at most and this value has been shown to diminish over time.

Shaped haptics, levers, or other mechanical elements have been described to translate the compressive force exerted by the elasticity of the lens capsule and/or the radial compressive force exerted by the ciliary muscles to effect a desired axial displacement of the IOL optic. Additional examples may also provide flexible hinge regions of the haptic to facilitate axial displacement of an IOL along the optical axis. Several examples include annular ring elements in contact with the lens capsule and that use the compression of applied force by the capsule to effect axial displacement of the IOL optic. However, these IOLs are configured to be generally of fixed optical power and in line with the optical axis of the eye. As such, the axial displacement of the optical elements of these IOLs that is possible limits the dioptric power change attained. Some single or multiple optic lenses have incorporated a shape changing and axial displacement changing combination of lenses, such as a shape changing optic coupled to zonular contact haptics whereby compression of the lens capsule during accommodation results in both anterior displacement of the flexible optic along the optical axis, as well as compression of the sides of the optic at the equator. Other described IOLs rely on a posterior flexible region separated from a flexible anterior lens by an articulating member about the circumference.

It is known that the lens capsule, following cataract surgery, becomes less pliable and more fibrotic. IOLs that rely on retained capsular elasticity/pliability are unlikely to retain accommodating/dis-accommodating ability.

Surface shape changing lenses are more likely to result in greater degrees of dioptric power change. These lenses include lenses with fluid filled chambers that rely on compression by the lens capsule along the optical axis to force fluid from a peripheral chamber into a central lens and thereby change the shape of the central lens and therefore the optical power of the lens. Other lenses use the compressive force by the lens capsule to provide a radial inward compressive force about the equatorial periphery of a flexible lens to shape change the lens. These are generally two-part systems with a circumferential haptic design with a central fixed posterior lens that fits within the capsule and then a separately placed pliable optic secured within the outer haptic ring. Compression by the “elastic” lens capsule is meant to provide a compressive force to the central lens flexible optic. This radial force is applied to the equatorial region of the central lens flexible optic. Other IOLs use a compressive force exerted on rigid haptics to compress a pliable optic against a separate fixed power posterior lens. These IOLs rely on the shape change of the posterior surface of the pliable optical element pressed against a fixed optical element or pressed against a relatively rigid posterior lens capsule to alter the dioptric power of the lens system. Other IOLs incorporate a skirt with a capsular contact ring. Such IOLs rely on compression exerted by the “elastic” lens capsule to impart a compressive force on a capsular contact ring and the mechanical design of this ring pulls radially about the equator of the IOL's flexible optic. Again, because these IOLs rely on retained capsular elasticity/pliability and because it is generally known that the lens capsule following cataract surgery becomes less pliable and more fibrotic, it is unlikely these lenses will retain accommodating/dis-accommodating ability. None of these IOL designs selectively apply a radial force anterior or posterior to the equator of the flexible optic. None of the shape changing accommodating IOLs described above mimic the natural human lens during accommodation or effectively account for the inevitable loss of capsular elasticity/pliability and progressive fibrosis and stiffening of the lens capsule.

### SUMMARY

The present disclosure relates to ophthalmic devices including IOLs and more particularly to accommodating intraocular lenses (accommodating IOLs). In an aspect, a two-piece IOL is provided comprising a base and an anterior shape-changing exchangeable optic. The base can comprise a plurality of actuating haptics, each having a lateral end, a medial end, and an intermediate section extending between the lateral end and the medial end. The anterior optic can comprise an elastic anterior face located anterior to the equator and having a periphery, a plurality of anterior arms or attachment points or members interacting with the plurality of actuating haptics, the attachment points or members located about the periphery or extending from the periphery of the anterior face and releasably connected to the medial end of a respective one of the plurality of actuating haptics. The anterior optic can also include a posterior face, an elastic side wall extending from the anterior face to the posterior face, and a chamber located between the anterior face and the posterior face and containing a material.

### BRIEF DESCRIPTION OF THE DRAWINGS

FIG. 1 is a side view of a two-piece IOL according to an aspect of the present disclosure.

FIG. 2 is a top view of a two-piece IOL according to an aspect of the present disclosure.

### DETAILED DESCRIPTION

The present disclosure relates to an IOL such as, for example, an accommodative IOL. As used herein with respect to a described element, the terms “a,” “an,” and “the” include at least one or more of the described element(s) including combinations thereof unless otherwise indicated. Further the term “a,” “an,” and “the” can refer to one component performing a described functionality or more than more than component performing the same functionality. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. As such “substantially” refers to the complete or nearly complete extent of a characteristic, property, state, or structure. The exact allowable degree of deviation from the characteristic, property, state, or structure will be so as to have the same overall result as if the absolute characteristic, property, state, or structure were obtained. As used herein, the terms “anterior,” “posterior,” “superior,” “inferior,” “lateral,” and “medial” refer to the position of elements when a patient is in a standard anatomical position unless otherwise indicated. The terms “left,” “right,” “top” and “bottom” refer to the position of elements as they are depicted in the drawings and the terms “left” and “right” can be interchanged unless indicated otherwise. The terms “first,” “second,” etc. are used to distinguish one element from another and not used in a quantitative sense unless indicated otherwise. Thus, a “first” element described below could also be termed a “second” element. A component operably coupled or connected to another component can have intervening components between the components so long as the IOL can perform the stated purpose. By “integral” or “integrated” is meant that the described components are fabricated as one piece or multiple pieces affixed during manufacturing or the described components are otherwise not separable using a normal amount of force without damaging the integrity (i.e. tearing) of either of the components. A normal amount of force is the amount of force a user would use to remove a component meant to be separated from another component without damaging either component. As used herein a “patient” includes a mammal such as a human being. All IOLs as described herein are used for medical purposes and are therefore sterile. Components of IOLs as described herein can be used with IOLs described herein as well as other IOLs. For example, an IOL as described herein can be placed anterior to an existing, previously placed IOL. IOLs include fixed power, multifocal, EDOF, diffractive and other variable focus lenses. Although the drawings show certain elements of an IOL in combination, it should be noted that such elements can be included (or excluded) in other embodiments or aspects illustrated in other drawings or otherwise described in the specification. In other words, each of the disclosed aspects and embodiments of the present disclosure may be considered individually or in combination with other aspects and embodiments of the disclosure including patent applications incorporated by reference herein.

Unlike shape changing accommodating IOLs described by way of background, IOLs are provided herein that can mimic the gradient elastic properties of a natural youthful human lens during accommodation and include a shape-changing optic where components of the optic change shape as the IOL transitions from an accommodated state to a dis-accommodated state and vice versa. Without wishing to be bound by a specific mechanism of action, it is considered by some that the lens capsules' “elasticity” controls and shapes the lens as a whole (the lens nucleus and cortex). On this basis, the lens contents are considered pliable. However, the volume of the lens contents compared to the thickness and known modulus of elasticity of the lens capsule predicts that the lens capsule cannot solely control and alter the shape of the lens nucleus and cortex. Finite element analysis (FEA) predicts that radial tension about the equatorial region of a lens capsule filled with a soft pliable solid or liquid does not result in significant shape change to either the anterior or posterior surface of the lens compared to what is known to occur with the natural youthful human lens. It has been shown that application of a radial force anterior or posterior to the equator of a flexible optic results is significantly greater dioptric power change than application of that force about the equator. In other words, providing radial tension directed specifically to at least the anterior face of an accommodating IOL; having that tension directed at points anterior to the equator of the IOL; the anterior face of the IOL being more resistant to deformational change than the content(s) of a chamber underlying the anterior face; the anterior face demonstrating elastic properties in so much as the anterior face deforms when a force is applied to the anterior face and the anterior face will return to its original shape with the removal of the force, results in a greater amount of anterior face shape change and therefore accommodating dioptric power change than can be achieved with a similar force applied at points at or more near the equator of the IOL (e.g. equatorial). In addition, a force applied to the anterior face at points anterior to the equator of the IOL requires less diameter change of the anterior face per diopter of power change of the IOL compared to a similar force applied at points at or more near the equator of the IOL thereby allowing the anterior face of the IOL to shape change even with very small amounts of anterior face diameter change when going from an accommodated state, a dis-accommodated state, and states in between.

In particular, in an aspect, an IOL comprising a shape changing optic that can assume an accommodated state, a dis-accommodated state, and states therebetween is provided. Components of the shape-changing optic can be deformable such that radially directed ocular compression force(s) or tensile force(s) applied to the optic caused by ciliary muscle contraction or relaxation causes one or more components of the optic to change shape and allows the optic to change dioptric power. None of the described IOLs above apply a radially outward tensile force that is directly transferred to the anterior surface at point(s) anterior to the equator of the optic.

As such, components of a shape-changing optic can deform or change shape when a force is applied. If a component is less resistant to deformational change than another component, the former component is more likely to, or to a greater degree, deform for a given amount of applied or removed force than the latter component. A component is more resistant to deformational change than another component, if the former component is less likely to, or to a lesser degree, deform for a given amount of applied or removed force than the latter component. It is understood that for any given component resistant to deformational change, the force applied/removed to such component does not exceed the force that results in breakage of the component such that it is no longer useful for its therapeutic purpose.

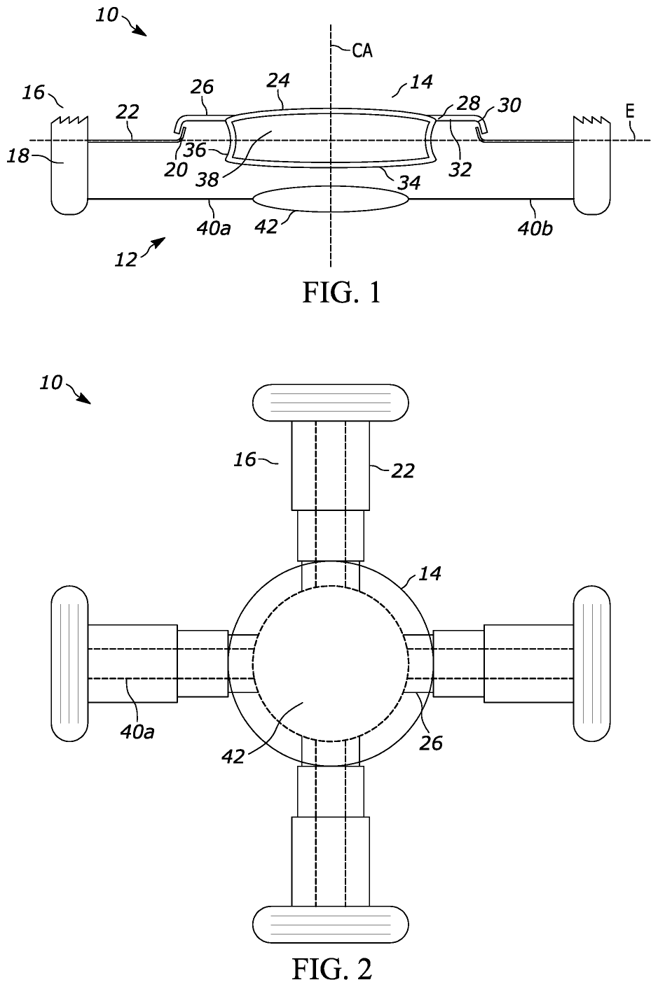

With reference to an exemplary IOL, FIG. 1 depicts a central or optical axis CA extending in an anterior-posterior direction and an equator E extending in a plane substantially perpendicular to the central axis. The equator is an imaginary line drawn around the circumference of a lens perpendicular to the optical axis, equally distant from the anterior face of the lens and the posterior face of the lens, dividing the lens into an anterior half and a posterior half. Referring to FIGS. 1-2, an IOL 10 is provided with an optical axis extending in an anterior-posterior direction, an equator extending in a plane substantially perpendicular to the optical axis, an accommodated state, a dis-accommodated state, and states therebetween. IOL 10 can comprise base 12, with or without an optic, and an anterior shape-changing exchangeable optic 14 releasably connected to base 12. By having the anterior optic releasably connected to the base, the anterior optic can be replaced or exchanged for another anterior optic having different optical properties.

In particular, base 12 can be configured to be placed in the capsular bag of a patient's eye and can comprise a plurality of actuating haptics 16 configured to response to radial forces applied by the ciliary muscle, via the lens zonules, to the lens capsule. Each of the plurality of actuating haptics 16 can have a lateral end 18, a medial end 20, and an intermediate section 22 extending between lateral end 18 and medial end 20. Although FIG. 2 illustrates only four actuating haptics, the base can comprise any suitable number of haptics, such as, for example, eight circumferentially extending haptics. Anterior shape-changing exchangeable optic 14 can comprise an elastic anterior face 24 located anterior to the equator and having a periphery. A plurality of anterior arms 26 (or other attachment member(s) (e.g. a ring) with attachment locations or points for the actuating haptics 16) can extend from the periphery of anterior face. In the case of a plurality of anterior arms, each arm can have a medial end 28 extending from anterior face 24, a lateral end 30 releasably connected to medial end 20 of a respective one of the plurality of actuating haptics 16, and an intermediate section 32 between medial end 28 and lateral end 30. The anterior shape-changing optic can be releasably connected to the base in a number of different ways. For example, the medial end of one or more of the plurality of actuating haptics or ring located about the periphery of the anterior surface of the anterior optic, and the lateral end of a respective one or more of the plurality of arms can comprise interlocking projections as shown in FIG. 1. Anterior shape-changing optic 14 can also have a posterior face 34 and an elastic side wall 36 extending from anterior face 24 to posterior face 34. A chamber 38 can be located between anterior face 24 and posterior face 34 and can contain a material as described in more detail below. The anterior optic can be spherical, aspheric, toric, multifocal, extended depth of focus, or have any other suitable optical properties, or combinations thereof. In certain aspects, the IOL can further comprise at least two opposing stabilizing, non-actuating haptics 40 connected to a respective lateral end 18 of at least two of the plurality of actuating haptics 16. A posterior optic 42 can be located between the at least two opposing stabilizing, non-actuating haptics (e.g. 40a and 40b). The posterior optic can be spherical, aspheric, toric, multifocal, extended depth of focus, or have any other suitable optical properties, or combinations thereof

Components of the shape-changing optic can be made to be more or less resistant to deformational change by altering the thickness of the component, the type of material from which the component is fabricated, or by altering the chemical/material properties of the component material itself, for example. In certain aspects, the anterior face of the anterior exchangeable optic is more resistant to deformational change than the material in the chamber.

Regarding specific components of an IOL, the anterior face, as stated above, can have elastic properties. Elastic properties can allow for the anterior face to change shape with an applied force, but also to return to its original configuration when the force is removed. It is beneficial that the anterior face be more resistant to deformational change (e.g. less pliable, firmer) than the contents or material contained within the chamber because when an outward radial force is applied to the anterior face, the contents of the chamber can more easily deform to allow flattening of the anterior face. Exemplary fabrication materials for the anterior face include silicone, an acrylic (hydrophobic or hydrophilic) polymer, polymethylmethalcryalate (PMMA), silastic, collamer, a suitable optical thermoplastic polymer, another suitable optical material, and suitable combinations thereof. The anterior face and/or the posterior face can comprise a lens with a variety of optical properties, such as, for example, a spherical, aspheric, toric, toroidal, multifocal, diffractive, extended depth of focus, or combinations thereof.

Regarding the side wall, as stated above, the side wall can have elastic properties. In certain aspects, the side wall can be fabricated from a material that is equal to or less resistant to deformational change than the anterior face. Such features can allow for the contents contained within the chamber to expand the area of the side wall to allow the volume of the contents of the chamber to remain the same when the anterior surface is flattened. Having the side wall deform can facilitate and allow for a greater amount of shape change to the anterior face of the shape-changing optic. Exemplary fabrication materials for the side wall include silicone, an acrylic (hydrophobic or hydrophilic) polymer, polymethylmethalcryalate (PMMA), silastic, collamer, a suitable optical thermoplastic polymer, another suitable material, or a suitable combination thereof. The side wall can also be equal to or less resistant to deformational change than the anterior face or the posterior face by being thinner than the anterior face or the posterior face.

Regarding the chamber, the chamber can be defined by the posterior surface of the anterior face, the anterior surface of the posterior face, and an inner surface of the side wall. The interior contents or material of the chamber can comprise a soft solid, a gel, a viscoelastic material, a flowable fluid, or a gas, or other suitable material. Exemplary materials that can be contained within the interior of the chamber include a soft silicone, or other soft material subject to deformational change, air or other gas, silicone oil (of various refractive indices), an aqueous solution of saline or hyaluronic acid, a viscoelastic polymer, polyphenyl ether, or other optical fluid, solid or gases, or suitable combinations thereof. The chamber can have an internal layer or coating, such as a parylene coating for example, to seal the contents of the chamber from the anterior face, the side wall and/or the posterior face. The chamber can be pre-loaded (e.g. by a manufacturer) with a suitable material. Alternatively, the chamber can be loaded with a suitable material by a clinician.

Regarding the plurality of actuating haptics of the base, such actuating haptics are the portion of the IOL that are configured to interact with the lens capsule, the lens zonules, the ciliary muscle, or other parts of a patient's eye. The shape-changing optic can change shape in response to an ocular force, specifically a force generated by the contraction or relaxation of the ciliary muscle of the patient's eye. The plurality of actuating haptics, interacting with the lens capsule, can apply radial outward tension to the anterior face of the anterior optic when the ciliary muscle relaxes and radial outward tension is placed on the lens capsule via the lens zonules. The plurality of actuating haptics can be elastic but can be more resistant to deformational change than the anterior face of the optic. An advantage to this is that the actuating haptics can be firmer to provide a linear, radially directed force from the actuating haptics that is directly transferred to the periphery of the anterior face. Without wishing to be bound by any particular mechanism of action, if the actuating haptics were less resistant to deformational change than the anterior face, the radial tension could result in stretching of the actuating haptics and less tension on the periphery of the anterior face. Thus, the anterior face may not shape change as much for a given force applied to the haptics. Exemplary fabrication materials for the actuating haptics include silicone, an acrylic (hydrophobic or hydrophilic) polymer, polymethylmethalcryalate (PMMA), silastic, collamer, a suitable optical thermoplastic polymer, another suitable material, or suitable combinations thereof.

When implanted and when the ciliary muscles of a patient's eye relax (such as when the eye is in a dis-accommodated state), the ciliary muscles apply tensile force to the plurality of actuating haptics (via the lens capsule with lens zonule attachments between the lens capsule and the ciliary muscles, for example). The plurality of actuating haptics, in turn, can apply tensile force to the periphery of the anterior face of the anterior optic at each site (referred to herein as a “connection site”) where a respective actuating haptic releasably connects with an anterior arm or other attachment member(s) such as, for example a ring of the anterior face of the anterior optic. The net result can be that the anterior face of the anterior optic can be pulled radially outward substantially perpendicular to the optical axis from several connection sites and functionally result in relatively symmetric radial tension placed on the periphery of the anterior face of the anterior shape-changing optic. The plurality of actuating haptics can engage the inner surface of the lens capsule or the outer surface of the lens capsule.

Further regarding the actuating haptics, in certain aspects, each of the plurality of actuating haptics can be non-rotatable in response to axial compression along the optical axis on the shape-changing optic. In certain aspects, each of the plurality of actuating haptics has a peripheral portion having a posterior face and an anterior face, with the posterior face being curved. In other aspects, the medial portion of each of the plurality of actuating haptic is releasably connected to each of the respective plurality of anterior arms or peripheral ring, for example, of the anterior face of the anterior optic such that the plurality of actuating haptics changes the shape of the anterior face of the anterior optic via application of radial tension to the periphery of the anterior face in a direction perpendicular to the optical axis. For example, the shape change of the anterior face is not via compressive forces along the optical axis on the shape-changing optic.

Regarding the stabilizing, non-actuating haptics, the stabilizing haptics can facilitate the proper positioning of the actuating haptics in the periphery of the lens capsule. The stabilizing haptics can also facilitate proper centration of the base and a base optic, if present.

The posterior optic (in embodiments including a posterior optic) can comprise a fixed power, multifocal, EDOF, diffractive or other variable focus lenses. The posterior lens can have a variety of optical properties, such as, for example, a spherical, aspheric, toric, toroidal, multifocal, diffractive, extended depth of focus, or combinations thereof.

In certain aspects, the actuating haptics, the non-actuating haptics and/or the arms of the optic can contain a therapeutic agent. Non-limiting examples of therapeutic agents include an intraocular steroid, an antibiotic or combinations thereof, to mitigate post-operative inflammation/infection such that pharmacologic eye drops or periocular injections may not be necessary, a therapeutic agent for improving glaucoma or macular degeneration, or combinations thereof. For example and with respect to chronic conditions such as glaucoma or macular degeneration, the therapeutic agent can be placed in the recessed areas of the haptics (in embodiments having such recessed areas) for long-term of sustained release of the therapeutic agent.

Each of the disclosed aspects and embodiments of the present disclosure may be considered individually or in combination with other aspects and embodiments as well as with respect to other intra-ocular lenses, such as IOLs disclosed in U.S. patent application Ser. No. 16/288,723 filed on Feb. 28, 2019 and incorporated by reference in its entirety and U.S. Provisional Application No. 62/842,788 filed on May 3, 2019 and incorporated by reference in its entirety. In addition, orientations of a shape-changing optic can be modified. For example, when implanted, the lens can be flipped such that the anterior face is facing in a posterior direction and the posterior face is facing in an anterior direction. Further, the IOL can be configured such that it is foldable for insertion. Further, while certain features of embodiments may be shown in only certain figures, such features can be incorporated into or deleted from other embodiments shown in other figures or otherwise disclosed in the specification. Additionally, when describing a range, all points within that range are included in this disclosure.

What is claimed is:

1. A two-piece intraocular lens (IOL) having an optical axis extending in an anterior-posterior direction, an equator extending in a plane substantially perpendicular to the optical axis, an accommodated state, a dis-accommodated state, and states therebetween, the IOL comprising:
a base comprising a plurality of actuating haptics, each having a lateral end, a medial end, and an intermediate section extending between the lateral end and the medial end; and
an anterior shape-changing exchangeable optic comprising:
an elastic anterior face located anterior to the equator and having a periphery;
an attachment member located about the periphery or extending from the periphery and releasably connected to the medial end of at least some of the plurality of actuating haptics;
a posterior face;
an elastic side wall extending from the anterior face to the posterior face; and
a chamber located between the anterior face and the posterior face and containing material.

2. The IOL of claim 1, wherein the attachment member comprises:
a plurality of anterior arms extending from the periphery of the anterior face and each having a medial end extending from the anterior face and a lateral end releasably connected to the medial end of a respective one of the plurality of actuating haptics, and an intermediate section between the medial end and the lateral end.

3. The IOL of claim 1, wherein the anterior face is more resistant to deformational change than the material.

4. The IOL of claim 1, further comprising at least two opposing stabilizing, non-actuating haptics connected to a respective lateral end of at least two of the plurality of actuating haptics.

5. The IOL of claim 4, further comprising a posterior optic located between the at least two opposing stabilizing, non-actuating haptics.

6. The IOL of claim 5, wherein the posterior optic comprises a spherical, aspheric, toric, toroidal, multifocal, diffractive, extended depth of focus lens, or combinations thereof.

7. The shape-changing optic of claim 5, wherein the posterior optic comprises a fixed power(s) lens.

8. The IOL of claim 2, wherein the medial end of one or more of the plurality of actuating haptics and the lateral end of a respective one or more of the plurality of anterior arms comprises interlocking projections to releasably attach the medial end of the one or more plurality of actuating haptics and the lateral end of the respective one or more of the plurality of arms.

9. The IOL of claim 4, wherein at least one or more of the plurality of actuating haptics, the attachment member, one or more of the stabilizing haptics, or combinations thereof comprises a therapeutic agent.
